(19) United States
(12) Patent Application Publication
(10) Pub. No.: US 2024/0180479 A1
(43) Pub. Date: Jun. 6, 2024

(54) Treatment of Parkinson's Disease

(71) Applicants: PROTHENA BIOSCIENCES LIMITED, Dublin2 (IE); HOFFMANN-LA ROCHE INC., Little Fall, NJ (US)

(72) Inventor: Gennaro Pagano, Basel (CH)

(21) Appl. No.: 18/279,408

(22) PCT Filed: Mar. 8, 2022
(86) PCT No.: PCT/US2022/019233

§ 371 Date: Aug. 30, 2023

Related U.S. Application Data
(60) Provisional application No. 63/158,239, filed on Mar. 8, 2021.

(51) Int. Cl. A61B 5/00; A61B 5/11; A61K 39/00; A61K 45/06; A61P 25/16; C07K 16/18

(52) U.S. Cl. CPC A61B 5/4082; A61B 5/1101; A61B 5/112; A61B 5/1124; A61B 5/4848; A61K 45/06; A61P 25/16; C07K 16/18; A61K 2039/505; A61K 2039/545

(57) ABSTRACT

The invention provides a method of treating, preventing or ameliorating Parkinson's disease with Prasinezumab; wherein providing the patient with a mobile device programmed to receive and transmit data acquired to measure passive and/or active movement of the patient (b) collecting data transmitted from the mobile device; and (c) comparing the data acquired from the patient with control data to assess presence or extent of movement deficits in the subject and/or monitoring the data acquired from the patient for a period of time sufficient to identify changes in the patient's active or passive motor function.

### CROSS REFERENCE TO RELATED APPLICATIONS

This application claims priority to U.S. Provisional Application U.S. Provisional Application No. 63/158,239, filed Mar. 8, 2021, the disclosure of which is incorporated by reference in its entirety.

### SEQUENCE LISTING

A computer readable form of the Sequence Listing is filed with this application by electronic submission and is incorporated into this application by reference in its entirety. The Sequence Listing is contained in the ASCII text file created on Mar. 3, 2022, having the file name “20-1293-WO2_Sequence-Listing_ST25.txt” and is 15 kb in size.

### BACKGROUND

Parkinson's disease (PD) is a slow, chronic, progressive neurodegenerative estimated to affect between 7-10 million people worldwide. In the United States, an estimated 725,000 people are affected and more than 50,000 new cases are reported every year. Although 5 to 10 percent of patients are diagnosed before age 50, PD is generally considered a disease that targets older adults, affecting one out of every 100 people over the age of 60, and it is more common in men than in women.

Alpha-synuclein is a protein that is normally associated with synapses and is believed to play a role in neural plasticity, learning and memory. Alpha-synuclein can aggregate to form insoluble fibrils in pathological conditions, and is a major component of pathology that characterizes several neurodegenerative disorders including Parkinson's disease. Soluble oligomers of alpha-synuclein may be neurotoxic. The accumulation of alpha-synuclein with similar morphological and neurological alterations in species and animal models as diverse as humans, mice, and flies suggests that this molecule contributes to the development of Parkinson's disease. Antibodies directed against alpha-synuclein may be able to reduce alpha-synuclein deposits and symptoms in Parkinson's disease.

Current treatments for PD manage the early motor symptoms of the disease, mainly through the use of dopamine replacement therapy and dopamine receptor agonists. Treatment with levodopa and other dopaminergic agents temporarily addresses the motor symptoms. However, this does not reverse, slow, or halt pathological processes related to the disease. As the disease progresses, these drugs become less effective at controlling the symptoms.

Patients who take these medications often develop side effects such as motor complications (e.g., response oscillations, wearing off phenomena, and drug-induced dyskinesias), as well as nausea, daytime somnolence, sleep attacks, orthostatic hypotension, or impulse control disorders. Symptomatic treatment of non-motor symptoms of PD (e.g., sleep disturbances, anxiety, and depression) are also available. However, to date, there are no approved treatments that have demonstrated protection of neurons or modification of the disease course. There is an urgent need for new therapies that target the underlying cause of Parkinson's disease and, unlike symptomatic therapies, slow its relentless progression.

### SUMMARY

In one aspect, the disclosure is directed to a method for monitoring the motor function of a patient that has Parkinson's Disease (PD) or is at risk for PD who has been administered Prasinezumab. The method may include:
(a) providing the patient with a mobile device programmed to receive and transmit data acquired from sensors internal and/or external to the mobile device that measure passive and/or active movement of the patient or a mobile device application programmed to receive and transmit data acquired from sensors internal and/or external to the mobile device that measure passive and/or active movement of the patient;
(b) collecting data transmitted from the mobile device; and
(c) comparing the data acquired from the patient with control data to assess presence or extent of movement deficits in the subject and/or monitoring the data acquired from the patient for a period of time sufficient to identify changes in the patient's active or passive motor function.

In various aspects of the disclosure, the sensors measure one or more of the following features of the patient's movement:
(a) median gesture power of passively monitored gestures:
(b) median turn speed in U-turn test and passively monitored gait,
(c) jerk in balance test,
(d) mel frequency cepstrum 2 in speech test,
(e) voice jitter in sustained phonation,
(f) number correct in Symbol Digit Modalities Test.
(g) speeded tapping variability,
(h) maximum speed of hand-turning,
(i) spiral celerity in draw-a-shape task, and
(j) median squared energy in rest and postural tremor tasks.
   The sensors may independently measure movement from the least affected side and the most affected side of the patient.

In another aspect of the disclosure, the data collected from the device is compared the patient's MDS-UPDRS score, for example UPDRS Part III.

The method of the disclosure may include administering a regimen of Prasinezumab to a patient. A Prasinezumab regimen according to the disclosure may include treatment of the patients with 1000-5000 mg of Prasinezumab at intervals of 3 to 5 weeks, and the treatment may further include administering to the patient a MAO-B inhibitor.

In a further aspect of the disclosure, period of time sufficient to identify changes in the patient's active or passive motor function comprises 4-52 weeks.

### DESCRIPTION OF THE DRAWINGS

FIG. 1 shows the change in total MDS-UPDRS score (Parts I, II, and III) from baseline to Week 52. Patients who started symptomatic PD treatment contribute until the last visit before symptomatic PD treatment is started. The results show that change from baseline in the MDS-UPDRS total score (Parts I, II, and III) at 52 weeks in each treatment group versus the placebo group was not met (pooled dose levels: −14.0%, −1.30, 80% CI=(−3.18, 0.58); low dose level: −21.5%, −2.02, 80% CI=(−4.21, 0.18); and high dose level: −6.6%, −0.62, 80% CI=(−2.82, 1.58)). Bars represent 80% CI. MDS-UPDRS, Movement Disorder Society Unified Parkinson's Disease Rating Scale.

FIG. 2A shows a change in total MDS-UPDRS Part III from baseline to Week 52 for site rating confirming a reduced decline in motor function (pooled dose levels: −25.0%, −1.44, 80% CI=(−2.83, −0.06); low dose level: −33.8%, −1.88, 80% CI=(−3.49, −0.27); and high dose level: −18.2%, −1.02, 80% CI=(−2.64, 0.61)). *Patients who started symptomatic PD treatment contribute until the last visit before symptomatic PD treatment is started. Bars represent 80% CI.

FIG. 2B shows a change in total MDS-UPDRS Part III from baseline to Week 52 for central rating confirming a reduced decline in motor function (pooled dose levels: −35.0%, −1.88, 80% CI=(−3.31, −0.45); low dose level: −45.4%, −2.44, 80% CI=(−4.09, −0.78); and high dose level: −24.7%, −1.33, 80% CI=(−2.99, 0.34)). Prasinezumab reduced decline in motor function by 35% vs. placebo after one year of treatment on the centrally rated assessment of MDS-UPDRS Part III. *Patients who started symptomatic PD treatment contribute until the last visit before symptomatic PD treatment is started. Bars represent 80% CI.

FIG. 3 shows there is a reduced time to worsening of motor function with delay of progression to clinically meaningful decline. Prasinezumab delayed time to clinically meaningful worsening of motor progression in prasinezumab-treated patients vs. placebo over 52 weeks as demonstrated by site rating of time to at least a 5-point progression in MDS-UPDRS Part III (pooled dose levels: HR=0.82, 80% CI=0.64 to 0.99; low dose level: HR=0.77, 80% CI=0.63 to 0.96; and high dose level: HR=0.87, CI=0.70 to 1.07). *Wald CI/test. Pooled dose analysis is a post-hoc analysis. CI, confidence interval; MDS-UPDRS, Movement Disorder Society Unified Parkinson's Disease Rating Scale.

FIG. 4 shows a reduction in progression of bradykinesia from baseline to Week 52 confirming that there is a clinical decline in bradykinesia progression. Signals of efficacy were observed on change from baseline on bradykinesia in prasinezumab-treated patients vs. placebo at 52 weeks by site rating (pooled dose levels: −27.0%, −0.75, 80% CI=(−1.62, 0.11); low dose level: −38.3%, −1.07, 80% CI=(−2.07, −0.07); and high dose level: −15.7%, −0.44, 80% CI=(−1.45, 0.56)). Pooled dose analysis is a post-hoc analysis. CI, confidence interval; MDS-UPDRS, Movement Disorder Society Unified Parkinson's Disease Rating Scale.

FIG. 5 shows patient movement data for features having an FDR or less than or equal to two collected over two week periods for 52 weeks using a smart-phone app. Figure shows the mentoring results for speed tapping variability on the least affected side.

FIG. 6 shows patient movement data for features having an FDR or less than or equal to two collected over two week periods for 52 weeks using a smart-phone app. Figure shows the results of passively monitoring hand gesture power.

FIGS. 7A-7C show a slowing of clinical decline with prasinezumab was more evident in individuals with faster progression, as assessed by digital motor measures.

### DESCRIPTION

The fluctuating nature of Parkinsonian symptoms make it difficult to measure potential treatment effects from infrequent clinic visit data. Therefore, Digital Health Technology Tools (DHTTs) of the disclosure enable remote and therefore frequent assessments of a patient's disease, disease progression and treatment response.

The disclosure is directed to the methods and devices for the measurement of Parkinson's disease progressing and treatment response using a wearable or hand-held device that can measure a patient's motor function by using sensors on the device that are highly sensitive to motor manifestations. Devices include, but are not limited to, smart-phones and smart watches including applications allow for the monitoring and tracking of patient movement using an accelerometer, gyroscope or similar movement detection hardware and accompanying software. The devices and methods of the disclosure allow for ecological validity of the assessments because the device can measure patient movement in environments where patients live, work, and socialize in order to provide a continuous collection and assessment of data during a patient's normal routines.

The methods and devices of the disclosure can be used in conjunction with Prasinezumab, and other similar anti-alpha-synuclein humanized antibodies, in the treatment, prevention, and/or amelioration (e.g., reduction in disease progression) of Parkinson's disease, including early stage Parkinson's disease. Prasinezumab is used to improve, maintain, or reduce decline in motor function in individuals with Parkinson's disease, which can be monitored with the methods and devices of the disclosure. In one aspect of the disclosure, one measure of motor function is the Movement Disorder Society-Unified Parkinson's Disease Rating Scale (MDS-UPDRS) Part III, a clinical examination of motor function. In another aspect of the disclosure, MDS-UPDRS Part III is a site rated assessment. In another aspect of the disclosure, MDS-UPDRS Part III is a centrally rated assessment. Motor symptoms associated with Parkinson's disease include slowness of movement (bradykinesia), tremor, changes in speech, facial expression, rigidity, and gait can be measured and monitored with the methods and devices of the disclosure. In one aspect of the disclosure, the measuring and monitoring can use used to show a delay in time to clinically meaningful worsening of motor progression on MDS-UPDRS Part III by treatment with Prasinuzemab.

Prior to addressing further aspects of the disclosure, a number of terms are defined below. As used herein, the singular forms “a,” “an”, and “the” include plural referents unless the context clearly dictates otherwise. For example, the term “a compound” or “at least one compound” can include a plurality of compounds, including mixtures thereof.

Alpha-synuclein is a highly conserved protein that is abundant in neurons, especially presynaptic terminals, and is believed to misfold and aggregate to form the protein structures that are highly implicated in Parkinson's disease pathology. Aggregated alpha-synuclein proteins form brain lesions are hallmarks of neurodegenerative synucleinopathies. Furthermore, misfolding and aggregation can often be accompanied by 0-amyloid deposition in some neurodegenerative diseases, and alpha-synuclein and tau aggregates coexist in several neurodegenerative disorders, including Parkinson's disease.

Natural human wild type alpha-synuclein is a peptide of 140 amino acids having the following amino acid sequence (GenBank accession number: P37840):

(SEQ ID NO: 8)
MDVFMKGLSK AKEGVVAAAE KTKQGVAEAA GKTKEGVLYV
GSKTKEGVVH GVATVAEKTK EQVTNVGGAV VTGVTAVAQK
TVEGAGSIAA ATGFVKKDQL GKNEEGAPQE GILEDMPVDP
DNEAYEMPSE EGYQDYEPEA.

The protein has three recognized domains: an-N-terminal repeat domain covering amino acids 1-61; a NAC (Non-amyloid component) domain running from about amino acids 60-95; and a C-terminal acidic domain running from about amino acid 98 to 140. Unless otherwise apparent from the context, reference to alpha-synuclein or its fragments includes the natural human wildtype amino acid sequences indicated above, and human allelic variants thereof, particularly those associated with Parkinson's disease.

Unless otherwise apparent from the context, the term “about” encompasses insubstantial variations, such as values within a standard margin of error of measurement (e.g., SEM) of a stated value. Designation of a range of values includes all integers within or defining the range, and all subranges defined by integers within the range. As used herein, statistical significance means p≤0.05. Unless otherwise apparent from the context, the term “about” encompasses values within the standard deviation of the mean of a stated value or +/−5% of a stated value, whichever is greater.

Compositions or methods “comprising” or “including” one or more recited elements may include other elements not specifically recited. For example, a composition that “comprises” or “includes” a polypeptide sequence may contain the sequence alone or in combination with other sequences or ingredients.

An individual is at increased risk of a disease if the subject has at least one known risk-factor (e.g., age, genetic, biochemical, family history, and situational exposure) placing individuals with that risk factor at a statistically significant greater risk of developing the disease than individuals without the risk factor.

The terms “subject” or “patient” include human and other mammalian subjects that receive either prophylactic or therapeutic treatment, including treatment naïve subjects. As used herein, the terms “subject” or “patient” refer to any single subject for which treatment is desired, including other mammalian subjects such as, humans, cattle, dogs, guinea pigs, rabbits, and so on. Also intended to be included as a subject are any subjects involved in clinical research trials not showing any clinical sign of disease, or subjects involved in epidemiological studies, or subjects used as controls. In some aspects of the disclosure, the patient is a male patient, and in some aspects of the disclosure, the patient is a female patient.

The term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The term “symptom” refers to a subjective evidence of a disease, such as altered gait, as perceived by the subject. A “sign” or “signal” refers to objective evidence of a disease as observed by a clinician or a physician.

As used herein, the terms “treat” and “treatment” refer to the alleviation or amelioration of one or more symptoms, signs, signals or effects associated with the disease, prevention, inhibition or delay of the onset of one or more symptoms or effects of the disease, lessening of the severity or frequency of one or more symptoms or effects of the disease, and/or increasing or trending toward desired outcomes as described herein. A treatment regimen refers to a combination of parameters characterizing administration of an antibody of the disclosure including any or all of dose, frequency of administration, route of administration, and total duration of administration.

The terms “prevention”, “prevent”, or “preventing” as used herein refer to contacting (for example, administering) the compositions of the present disclosure with a subject before the onset of a disease, with or without alpha-synuclein pathology already present (primary and secondary prevention), thereby delaying the onset of clinical symptoms and/or alleviating symptoms of the disease after the onset of the disease, compared to when the subject is not contacted with the peptide or immunotherapy compositions, and does not refer to completely suppressing the onset of the disease. In some cases, prevention may occur for limited time after administration of the peptide or immunotherapy compositions of the present disclosure. In other cases, prevention may occur for the duration of a treatment regimen comprising administering the peptide or immunotherapy compositions of the present disclosure.

The terms “reduction”, “reduce”, or “reducing” as used herein refer to decreasing or suppressing an increase in the measurement or evaluation of a symptom, sign, signal or effect associated with Parkinson's disease. In other embodiments terms “reduction”, “reduce”, or “reducing” as used herein refer to decreasing or suppressing an increase in the amount of alpha-synuclein present in a subject or in tissue of the subject, which encompasses decreasing or suppressing an increase in (e.g., decreasing the rate of increase) the amount of alpha-synuclein present, accumulated, aggregated, or deposited in the subject or tissue in the subject. In certain embodiments, the decrease in or suppression of an increase in (e.g., decreasing the rate of increase) the amount of alpha-synuclein present, accumulated, aggregated, or deposited in the subject refers to an amount of alpha-synuclein present, accumulated, aggregated, or deposited in the central nervous system (CNS) of the subject. In certain embodiments, the decrease in or suppression of an increase in (e.g., decreasing the rate of increase) the amount of alpha-synuclein present, accumulated, aggregated, or deposited in the subject refers to an amount of alpha-synuclein present, accumulated, aggregated, or deposited in the periphery (e.g., peripheral circulatory system) of the subject. In certain embodiments, the decrease in or suppression of an increase in (e.g., decreasing the rate of increase) the amount of alpha-synuclein present, accumulated, aggregated, or deposited in the subject refers to an amount of alpha-synuclein present, accumulated, aggregated, or deposited in the brain of the subject. In some embodiments, the alpha-synuclein reduced is the pathological form(s) alpha-synuclein (e.g., fibular alpha-synuclein inclusions, oligomeric or fibrillar alpha-synuclein conglomerates, and protofibrillar intermediates of alpha-synuclein oligomers). In yet other embodiments, pathological indicators of neurodegenerative disease and/or synucleinopathies are decreased.

Prasinezumab (PRX002/RG7935) is an immunoglobulin class G1 (IgG1) humanized monoclonal antibody (mAb) derived from murine parental antibody 9E4 and is directed against an epitope in the C-terminus of human α synuclein (amino acids 118-126). Prasinezumab binds in biochemical and biophysical experiments to both soluble and insoluble forms of human α-synuclein, and with a greater relative affinity/avidity to aggregated over monomeric forms of α-synuclein. In cell culture, prasinezumab effectively blocks the cell-to-cell transmission of a synuclein. Prasinezumab includes a heavy chain variable region of SEQ ID NO:1 and a light chain variable region of SEQ ID NO:4. Other exemplary humanized forms of the mouse 9E4 antibody including three exemplified humanized light chain mature variable regions (SEQ ID NOs:2, 3) and four exemplified humanized heavy chain mature variable regions (SEQ ID NOs:5, 6, 7). The exemplary light and heavy chain mature variable regions can be paired in any combination. See WO2019/064053, which is incorporated by reference herein in its entirety. As demonstrated herein, Prasinezumab is the first potentially disease-modifying, anti-alpha-synuclein antibody to demonstrate signals of efficacy on multiple clinical endpoints in patients with early Parkinson's disease.

MDS-UPDRS Part III is a clinical examination of motor function that assesses motor symptoms associated with Parkinson's disease. In one aspect, Prasinezumab can be used to reduce the decline of motor function in a subject having Parkinson's disease or at risk, which can be measured and monitored with the devices and methods of the disclosure.

The measuring and monitoring can begin before or during treatment with Prasinezumab and be used to show a decline in motor function, as measured by MDS-UPDRS Part III, by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%; 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, or 140% or by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 111%, at least 112%, at least 113%, at least 114%, at least 115%, at least 116%, at least 117%, at least 118%, at least 119%, at least 120%, at least 121%, at least 122%, at least 123%, at least 124%, at least 125%, at least 126%, at least 127%, at least 128%, at least 129%, at least 130%, at least 131%, at least 132%, at least 133%, at least 134%, at least 135%, at least 136%, at least 137%, at least 138%, at least 139%, or at least 140%.

In another aspect, the methods and devices of the disclosure can be used to measure and monitor whether Prasinezumab reduces decline in motor function by 35% versus placebo after one year of treatment on the centrally rated assessment of MDS-UPDRS Part III, and by 25% versus placebo after one year of treatment on the site rated assessment of MDS-UPDRS Part III. In addition, the devices and methods can be used to show that Prasinezumab can improve Bradykinesia, one of the cardinal symptoms of Parkinson's disease that is assessed as a component of the MDS-UPDRS Part III clinical motor examination.

In one aspect, the methods and devices of the disclosure can be used to determine whether Prasinezumab or other therapies maintain motor function or delay time to clinically meaningful worsening of motor progression in a subject having Parkinson's disease or at risk of Parkinson's. The devices and methods can measure or assist in measuring a reduction Parkinson's disease progression, e.g., delay time to clinically meaningful worsening of motor progression. A reduction in disease progression can be demonstrated, for example, by extending the time to at least a 5-point progression in MDS-UPDRS Part III.

In various aspects of the disclosure, a regimen of Prasinezumab includes 1000-5000 mg of Prasinezumab at intervals of 3 to 5 weeks.

In another aspect of the disclosure, the devices and methods can show and improvement in a patient's MDS-UPDRS Part III motor examination score and/or improvement of one or more of speech, facial expression, rigidity, finger tapping, hand movements, pronation-supination movements of hands, toe tapping, leg agility, arising from chair, gait, freezing of gait, postural stability, posture, body bradykinesia, tremor of hands, rest tremor amplitude, constancy of rest tremor, or Hoehn and Yahr Stage. Still further, the devices and methods of the disclosure can show an improvement in bradykinesia, for example by at least 10%, at least 15%, at least 20%, at least 25%, at least 30%, at least 35%, or at least 40% vs. placebo after one year of treatment. Measurement of motor function can also be determined by, for example, positive signals on motor function as determined by a digital motor score that includes a composite score built from 80% bradykinesia features and 20% resting tremor features, or similar combinations thereof.

Remote Monitoring of Motor Function in Parkinson's Disease Patients

Treatment with Prasinezumab therapy can be accompanied by monitoring a subject receiving treatment for changes in movement. The monitoring may include assessment of at least one feature of motor function before and after commencing treatment. The monitoring can indicate reduced movement deficits responsive to treatment, which may be relative to before beginning treatment or at least indicates a reduced rate of decline relative to the previous rate of decline in the subject or the rate of decline in control patients not receiving any immunotherapy. Separately, subjects can also be monitored for changes in autonomic dysfunction, gastrointestinal dysfunction, visual hallucination or one or more psychological symptom among other signs or symptoms.

Symptoms of subjects, for example motor symptoms such as tremor, rigidity and slowness of movement, may be monitored. Wearable systems or on-body sensors may be used to assess and quantify motor symptoms of subjects. “On-body sensors” may be used in a laboratory setting or in free-living conditions See S. Del Din, et al., J. of NeuroEngineering and Rehabilitation, 2016 13:46.

Subjects may be monitored using mobile-device-based monitoring. The mobile device may be a smartphone, smartwatch, wearable sensor, portable multimedia device or tablet computer. Built-in mobile-device sensors may be used to record daily activities of subjects. Subjects may carry a mobile-device to record their daily activities. Mobile-device-based assessments and sensors may be used for remote, passive monitoring of gait and mobility in subjects receiving treatment, for example for Parkinson's disease. (See e.g., Lipsmeier, F., et al. Mov Disord. 2017; 32 (suppl 2); W. Y. Cheng et al, 2017 IEEE/ACM International Conference on Connected Health: Applications, Systems and Engineering Technologies (CHASE), Philadelphia, P A, 2017, pp. 249-250). Sensor data may be analyzed by machine learning-based activity profiling. Gait and mobility may be compared to or correlated with the MDS-UPDRS that is used in clinics to evaluate Parkinson's disease severity.

Mobile-device-based monitoring may include (a) providing a patient with a mobile device programmed to receive and transmit data acquired from sensors internal and/or external to the device relating to movement deficits of a subject having or suspected of having a Parkinson's disease. As the subject undergoes a series of movements to reveal movement deficits, if present, the internal or external sensors of the device can acquire data relating to the movements.

Examples of internal or external sensors can include, for example, gyroscopes, accelerometers, gravimeters, cameras, passive infrared sensors, and/or other hardware and accompanying software. In some examples, for a particular sensor, the associated hardware can be located on or in the mobile device along with the accompanying software. In other examples, the associated hardware can be located remote from the mobile device, but can be in wired or wireless communication with the mobile device to facilitate an exchange of data between the sensor and the mobile device.

The acquired data may be collected and transmitted from the mobile device, which allows for comparing the data acquired from the subjects with control data to assess presence or extent of movement deficits in the subject. In some mobile-device-based monitoring, the mobile device is programmed to receive and transmit data from at least two external sensors attached to upper and lower limbs of the subject. In some mobile-device-based monitoring, the mobile device acquires data from sensors on the upper and lower limbs of the subject. In some mobile-device-based monitoring, the mobile device is carried by the subject and acquires data from an internal sensor. In some mobile-device-based monitoring, the series of movements includes tapping the device, sitting and standing.

The mobile device may transmit active or passive movement from the patient. Accordingly various aspects of the disclosure in include a method for monitoring motor function of a Parkinson's Disease (PD) patient in response to a Prasinezumab therapy. The method includes (a) treating patient with the Prasinezumab therapy; (b) providing the patient with a mobile device programmed to receive and transmit data acquired from sensors internal and/or external to the device that measure passive and/or active movement of the patient; (c) collecting data transmitted from the mobile device; and (d) comparing the data acquired from the patient with control data to assess presence or extent of movement deficits in the subject and/or monitoring the data acquired from the patient for a period of time sufficient to identify changes in the patient's active or passive motor function.

The passive or active movement data from the patient may include one or more of the following features of the patient's movement, which may be collected independently from the patient's least affected side and the most affected side, or both:
(a) median gesture power of passively monitored gestures:
(b) median turn speed in U-turn test and passively monitored gait,
(c) jerk in balance test,
(d) mel frequency cepstrum 2 in speech test,
(e) voice jitter in sustained phonation,
(f) number correct in Symbol Digit Modalities Test.
(g) speeded tapping variability,
(h) maximum speed of hand-turning,
(i) spiral celerity in draw-a-shape task, and
(j) median squared energy in rest and postural tremor tasks.

The movement data collected from the device may be compared to or correlated with the patient's MDS-UPDRS score, in particular one or more of MDS-UPDRS Part I, MDS-UPDRS Part II, or UPDRS Part III.

With the device, a patient's active or passive movement may be monitored over the course of several days, weeks, months or years in order to determine the effect of Prasinezumab therapy on a patient's motor function. For example, a period of time sufficient to identify changes in the patient's active or passive motor function may include a period of 4-52 weeks, such as 4 weeks, 8 weeks, 16 weeks, 20 weeks, 24 weeks, 28 weeks, 32 weeks, 36 weeks, 42 weeks, 46 weeks or 52 weeks, or longer.

### Diagnostic Criteria for Parkinson's Disease

The present methods are in general performed on subjects diagnosed with Parkinson's disease by a qualified health practitioner or are at elevated risk thereof compared with the general population as evidenced by genetic or biochemical markers, family history or prodromal symptoms of the disease. Such individuals include any who have received a prior prescription for treatment or prophylaxis of Parkinson's disease. Diagnosis of the Parkinson's disease synucleinopathy can be based on art-recognized criteria for possible or probable Parkinson's disease, such as those of DSM-V or DSM IV-TR, the Lewy Body dementia association, the Parkinson's disease society and the like. However, diagnosis can also be based on presence of any signs or symptoms of Parkinson's disease that lead a treating physician to conclude that a subject probably has Parkinson's disease. Exemplary criteria for diagnosing possible or probable PD are shown below.
Group A: resting tremor, bradykinesia, rigidity and asymmetric onset
Group B features: suggestive of alternative diagnoses
   Prominent postural instability in the first 3 years after symptom onset
   Freezing phenomena in the first 3 years
   Hallucinations unrelated to medications in the first 3 years
   Dementia preceding motor symptoms or in the first year
   Supranuclear gaze palsy (other than restriction of upward gaze) or slowing of vertical saccades
   Severe symptomatic dysautonomia unrelated to medications

Documentation of a condition known to produce parkinsonism and plausibly connected to the subject's symptoms (such as suitably located focal brain lesions or neuroleptic use within the past 6 months).

Criteria for possible diagnosis of Parkinson's disease include the following: at least 2 of the 4 features in Group A are present; at least 1 of these is tremor or bradykinesia and either none of the features in Group B is present or symptoms have been present for less than 3 years and none of the features in Group B is present to date; and either substantial and sustained response to levodopa or a dopamine agonist has been documented, or the subject has not had an adequate trial of levodopa or dopamine agonist.

Criteria for probable diagnosis of Parkinson's disease include the following: at least 3 or the 4 features in Group A are present, and none of the features in Group B is present and substantial and sustained response to levodopa or a dopamine agonist has been documented.

### Therapeutic Regimens

In therapeutic applications, an antibody is administered to a subject diagnosed with PD in a regime (dose, frequency and route of administration) known or suspected to be effective to ameliorate or at least inhibit further deterioration of at least one sign or symptom of the disease. In prophylactic applications, an antibody is administered to a subject at increased risk of a synucleinopathy but not yet having sufficient symptoms to be diagnosed with the disease in a regime known or suspected to be effective to inhibit or delay onset of at least one sign or symptom of the disease.

An exemplary dosage range for antibodies is from 3000 to 5000 mg of an antibody against alpha-synuclein administered intravenously at intervals of 3-5 weeks, such as every 4 weeks. In some subjects, the dosage is 3500-4500 mg every 3-5 weeks, such as every 4 weeks. Subjects can receive the same or different dosages as each other (e.g., depending on weight of the subject). In some methods, a subject receives one of two fixed dosages. For example, subjects with a weight less than 65 kg can receive 3500 mg and subjects with a weight greater or equal to 65 kg can receive 4500 mg. In some methods, the dosage range for at least some subjects lies within a range of 45-75 mg/kg, for example, 50-70 mg/kg, 45 mg/kg, 60 mg/kg or 65 mg/kg. Dosages are usually administered on multiple occasions with an interval of 3-5 weeks, such as every 28 days or four weeks, or every calendar month. Subjects can receive at least 6, 9, 12 or 18 dosages at such intervals, or can be dosed while symptoms of the conditions persist or for the remaining life of the subject. In some regimes, an initial loading dose of 2000 mg is administered followed by dosing within a range of greater or equal to 2000 mg but less than the intended target dose until the intended target dose is reached. For example, a subject can receive an initial loading dose of 2000 mg, followed by an up-titration to a 3500 mg dose or a 4500 mg dose. The up-titration can occur in a single subsequent dose or in gradual increases over several doses until a target dose or dose within a target range is reached. For example, a subject can receive an initial dose of 2000 mg followed by subsequent doses of 3500 mg. Alternatively a subject can receive an initial dose of 2000 mg followed by one or more subsequent doses at greater or equal to 2000 mg but less than 3500 mg, and subsequent doses at 3500 mg. Likewise a subject can receive an initial dose of 2000 mg followed by subsequent doses of 4500 mg. Alternatively, a subject can receive an initial dose of 2000 mg followed by subsequent doses at greater or equal to 2000 mg but less than 4500 mg and subsequent doses at 4500 mg. In some regimes a subject receives a dose of 3000-5000 mg antibody intravenously every four weeks for at least 52 weeks. In subjects receiving multi-dose regimes with the dose within a specified range, such as 3500-5000 mg, the subject can receive the same or different dose within the specified range on each dosing. In some regimes, a subject receives the same dose within a specified range at each dosing.

In another exemplary regime, a dose of 1300-1700 mg antibody is administered intravenously to a subject at intervals of 3-5 weeks. An exemplary dose is 1500 mg. Subjects can received a single fixed dose or two or more different dosages within this range, based on e.g., subject weight. Some subjects dosed within this range receive 18-25 mg/kg of antibody, for example, 20 mg/kg. As in other methods, the intervals can be 3-5 weeks, such as every 4 weeks or every calendar month. Subjects can receive at least 6, at least 9, at least 12, or at least 18 dosages, or can be dosed at such intervals while symptoms remain or for the remaining life of a subject.

Any of the treatment regimens can be accompanied by monitoring a subject receiving treatment for changes in movement and/or cognitive deficits. Preferably such monitoring includes at least one assessment before and after commencing treatment. Preferably the monitoring indicates reduced movement and/or cognitive deficits responsive to treatment, that is relative to before beginning treatment or at least indicates a reduced rate of decline relative to the previous rate of decline in the subject or the rate of decline in control patients not receiving any immunotherapy. Subjects can also be monitored for changes in autonomic dysfunction, gastrointestinal dysfunction, visual hallucination or one or more psychological symptom among other signs or symptoms.

The present regimes can be administered concomitantly with another agent effective in treatment or prophylaxis of the disease being treated. The other agent can be another immunotherapeutic agent described herein or other agent for treating Parkinson's disease including levodopa, benzaseride, carbidopa, dopamine agonists, non-ergot dopamine agonists, catechol-O-methyl (“COMT”) inhibitors such as, for example, entacopone or tolcopone, monoamine oxidase (“MAO”) inhibitors, such as, for example, rasagaline, amantadine, or anticholinergic agents can be used in combination with the present regimes. Some such other agents reduce one or more symptoms of the disease without affecting causative factors.

### EXAMPLES

### Example 1. Phase II Clinical Trial for Prasinezumab

A phase II trial was conducted for the alpha-synuclein antibody Prasinezumab on subjects with Parkinson's disease (PASADENA, NCT03100149). The trial has two treatment arms and one control arm. Subjects are randomized 1:1:1 into the arms, with N=316. The initial phase of the trial was a 52-week double blind treatment. During the initial phase of the trial, subjects did not receive other treatments Parkinson's disease (including symptomatic treatment). The subjects in one treatment arm received a fixed dose of 1500 mg antibody (low dose) intravenously every four weeks. The subjects in the other treatment arm received 3500 mg or 4500 mg of antibody (high dose) intravenously every four weeks depending on weight with subjects below 65 kg receiving the low dose and subjects at or above 65 kg receiving the high dose. The subjects in the second arm received a loading dosage of 2000 mg and optionally additional up titration dosages at 2000 mg or above until reaching the target dose of 3500 mg or 4500 mg. Dosing was continued for one year (52 weeks). The trial then has an extension period in which subjects initially in the placebo group received one of the two treatment regimens from the initial phase, and subjects from the treatment arms in the initial phase continued to receive the same treatment as previously. During the extension phase of the trial, subjects may have received systematic treatment with levodopa as well as the antibody subject of the trial, but did not receive other treatments for Parkinson's disease.

TABLE 1
Baseline characteristics of Patients in Phase II trial

| | Placebo | | Low dose | | High dose | | All patients | |
|---|---|---|---|---|---|---|---|---|
| | (n = 105)* | | (n = 105)* | | (n = 106)* | | (N = 306) | |
| Age, years | | | | | | | | |
| Mean (SD) | 59.9 | (8.7) | 60.3 | (8.8) | 59.4 | (9.8) | 59.9 | (9.1) |
| Gender | | | | | | | | |
| Male N (%) | 71 | (67.6) | 71 | (67.6) | 71 | (67.0) | 213 | (67.4) |
| Female N (%) | 34 | (32.4) | 34 | (32.4) | 35 | (33.0) | | |
| Weight (kg) | | | | | | | | |
| Mean (SD) | 75.74 | (14.48) | 78.02 | (13.66) | 76.17 | (13.03) | | |
| Disease duration | | | | | | | | |
| Mean (SD) | 9.9 | (6.8) | 10.2 | (6.3) | 10.1 | (6.5) | 10.1 | (6.5) |
| MAO-Bi | | | | | | | | |
| Yes | 38 | (36.2) | 38 | (36.2) | 39 | (36.8) | 115 | (36.4) |
| No | 67 | (63.8) | 67 | (63.8) | 67 | (63.2) | 201 | (63.6) |
| Sub-phenotypes | | | | | | | | |
| Diffuse malignant | 15 | (14.3) | 21 | (20.0) | 23 | (21.7) | 59 | (18.7) |
| Mild motor predominant | 39 | (37.1) | 28 | (26.7) | 39 | (36.8) | 106 | (33.5) |
| Intermediate | 51 | (48.6) | 56 | (53.3) | 44 | (41.5) | 151 | (47.8) |
| MDS-UPDRS total | | | | | | | | |
| Mean (SD) | 32.01 | (12.98) | 31.49 | (13.32) | 30.75 | (12.10) | 31.41 | (12.78) |
| MDS-UPDRS Part III | | | | | | | | |
| Mean (SD) | 21.54 | (9.11) | 21.90 | (9.14) | 20.97 | (8.81) | 21.47 | (9.00) |
| MDS-UPDRS Part II | | | | | | | | |
| Mean (SD) | 5.55 | (4.09) | 4.94 | (3.99) | 5.50 | (4.07) | 5.33 | (4.04) |
| MDS-UPDRS Part I | | | | | | | | |
| Mean (SD) | 4.91 | (3.71) | 4.64 | (4.16) | 4.27 | (3.57) | 4.61 | (3.83) |
| H&Y Stage category | | | | | | | | |
| I (%) | 20 | (19.0) | 29 | (27.6) | 29 | (27.4) | 78 | (24.7) |
| II (%) | 85 | (81.0) | 76 | (72.4) | 77 | (72.6) | 238 | (75.3) |
| MoCA total score | | | | | | | | |
| Mean (SD) | 27.83 | (2.01) | 27.97 | (1.94) | 27.81 | (2.15) | | |
| SCOPA-AUT | | | | | | | | |
| Mean (SD) | 7.68 | (5.36) | 7.95 | (5.83) | 8.52 | (5.93) | | |
| PDSS-2 | | | | | | | | |
| Mean (SD) | 9.41 | (6.41) | 8.57 | (6.21) | 8.49 | (5.94) | | |
| REM sleep behaviour | | | | | | | | |
| Positive (≥5) | 24 | (22.9%) | 34 | (32.4%) | 27 | (25.7%) | | |
| Negative (<5) | 81 | (77.1%) | 71 | (67.6%) | 78 | (74.3%) | | |
| SE-ADL category | | | | | | | | |
| Independent | 104 | (99.0%) | 104 | (100%) | 106 | (100%) | | |
| Not independent | 1 | (1.0%) | 0 | | 0 | | | |
| CGI-S | | | | | | | | |
| Mean (SD) | 3.05 | (0.63) | 3.05 | (0.63) | 3.06 | (0.64) | | |
| PDQ-39 | | | | | | | | |
| Mean (SD) | 10.08 | (7.06) | 9.59 | (7.54) | 9.36 | (6.82) | | |
| DaT-SPECT imaging | | | | | | | | |
| Mean (SD) | 1.06 | (0.30) | 1.04 | (0.33) | 1.08 | (0.34) | | |
| Patients with evaluable | 76 | (72.4) | 75 | (73.5) | 73 | (70.9) | 224 | (70.9) |
| data at Week 52^{†} | | | | | | | | |

*n represents number of participants contributing to summary statistics. Percentages are based on n.
^{†}Visits are time windowed. Non-evaluable data is considered as patient starting symptomatic PD treatment, an increase on MAO-Bi (if the patient was on MAO-Bi at baseline), or withdrawal from the study.
H&Y, Hoehn and Yahr; MAO-Bi, monoamine oxidase B inhibitor; MDS-UPDRS, Movement Disorder Society Unified PD Rating Scale; PD, Parkinson's disease; SD, standard deviation.

Prasinezumab was found to be generally safe and well tolerated, with the majority of adverse events reported as mild or moderate and similar across placebo and both treatment arms. The majority of reported Adverse Events (AE) (92%) were mild (grade 1-2). A single grade 4 AE was reported and deemed to be unrelated to study drug. There were no grade 5 AEs (see Table 2).

TABLE 2
Overview of Safety Data

| | Placebo | Low dose | High dose | All patients |
|---|---|---|---|---|
| | (n = 105) | (n = 105) | (n = 106) | (n = 316) |
| Total number of AEs* | 411 | 428 | 549 | 1388 |
| Total number of AE | 0 | 0 | 0 | 0 |
| with fatal outcome | | | | |
| (Grade 5)* | | | | |

Total number of patients with at least one (%):^{†}

| | | | | | | | | |
|---|---|---|---|---|---|---|---|---|
| AE | 87 | (82.9) | 98 | (93.3) | 97 | (91.5) | 282 | (89.2) |
| SAE | 5 | (4.8) | 7 | (6.7) | 8 | (7.5) | 20 | (6.3) |
| Grade 3-4 AE | 8 | (7.6) | 4 | (3.8) | 8 | (7.5) | 20 | (6.3) |
| AE leading to withdrawal | 1 | (0.9) | 2 | (1.9) | 5 | (4.7) | 8 | (2.5) |
| from treatment or dose | | | | | | | | |
| interruption | | | | | | | | |
| All Grade IRR | 17 | (16.2) | 20 | (19.0) | 36 | (33.9) | 73 | (23.1) |
| Grade 1-2 IRR | 17 | (16.2) | 20 | (19.0) | 35 | (33) | 72 | (22.8) |

| | | | | | | |
|---|---|---|---|---|---|---|
| Grade 3 IRR | 0 | 0 | 1 | (0.9) | 1 | (0.3) |

*Most AEs were Grade 1-2. Only one Grade 4 AE (suicide attempt) was reported and considered unrelated to study treatment (High-dose group). The most frequently reported (>1.0%) Grade 3-4 AEs were: radius fracture - two patients (1.9%) in the Placebo group (but no patient in the prasinezumab-treated groups) and anxiety - two patients (1.9%) in the High-dose group (no patient in the Low-dose or Placebo group).
^{†}Percentages are based on N in the column headings. Only treatment-emergent AEs are displayed where the study medication adjustment case report form question is answered as “drug withdrawn”.
For frequency counts by preferred term, multiple occurrences of the same AE in an individual are counted only once.
For frequency counts of “total number of events” rows, multiple occurrences of the same AE in an individual are counted separately.
AE, adverse event; IRR, infusion-related reaction; SAE, serious AE.

### Objectives:

The primary objective was to evaluate the efficacy of prasinezumab versus placebo at Week 52 in participants with early PD (H&Y Stages III) who are untreated or treated with MAO-B inhibitors since baseline, as measured by change from baseline on the MDS UPDRS Total Score (sum of Parts I, II and III).

Secondary objectives are to evaluate the effects of prasinezumab versus placebo at Week 52, in participants with early PD (H&Y Stages III) who are untreated or treated with MAO-B inhibitors since baseline, on the following:
MDS-UPDRS;
Dopamine transporter imaging with single photon emission computed tomography (DaT-SPECT) in the ipsilateral (to the clinically dominant side) putamen;
Montreal Cognition Assessment (MoCA) total score;
Clinical Global Impression of Improvement (CGI-I);
Patient Global Impression of Change (PGI-C);
Schwab and England Activity of Daily Living (SE-ADL) score;
Time to worsening in motor or non-motor symptoms; and/or
Time to start of dopaminergic PD treatment (levodopa or dopamine agonists).

### Example 2 Patients with Parkinson's Disease Treated with Prasinezumab Show Improvement in Motor Function

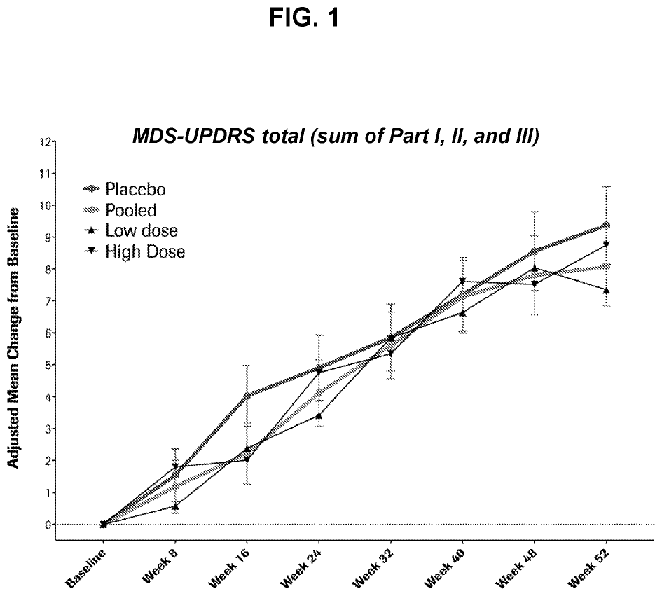

The study in Example 1 did not meet the primary endpoint of change in MDS-UPDRS total score (FIG. 1; −21.5% Low dose: −2.02 80% CI −4.21, −0.18; −6.6% High dose: −0.62 80% CI −2.82, −1.58). However, a surprising signal of efficacy was observed on change from baseline in MDS-UPDRS Part III in prasinezumab-treated patients versus placebo at 52 weeks. Prasinezumab-treated patients demonstrated reduced decline in motor function versus placebo at one year and delayed time to clinically meaningful worsening of motor progression in patients with early Parkinson's disease.

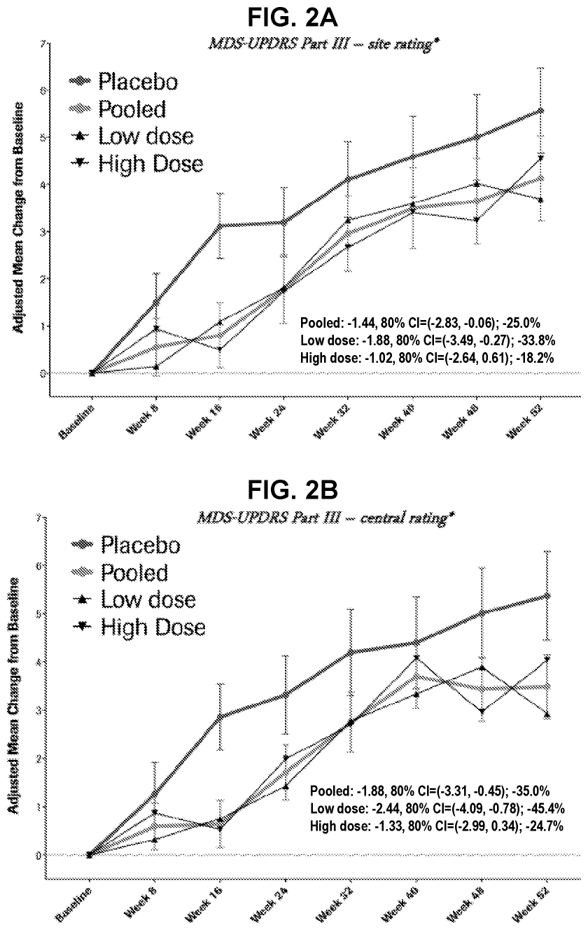

Using MDS-UPDRS Part III site ratings, patients demonstrate a reduced decline in motor function (FIG. 2A; pooled dose levels: −25.0%, −1.44, 80% CI=(−2.83, −0.06); low dose level: −33.8%, −1.88, 80% CI=(−3.49, −0.27); and high dose level: −18.2%, −1.02, 80% CI=(−2.64, 0.61)).

Prasinezumab also reduced decline in motor function by 35% versus placebo after one year of treatment on the centrally rated assessment of MDS-UPDRS Part III, a clinical examination of motor function (FIG. 2B; pooled dose levels: −35.0%, −1.88, 80% CI=(−3.31, −0.45); low dose level: −45.4%, −2.44, 80% CI=(−4.09, −0.78); and high dose level: −24.7%, −1.33, 80% CI=(−2.99, 0.34)).

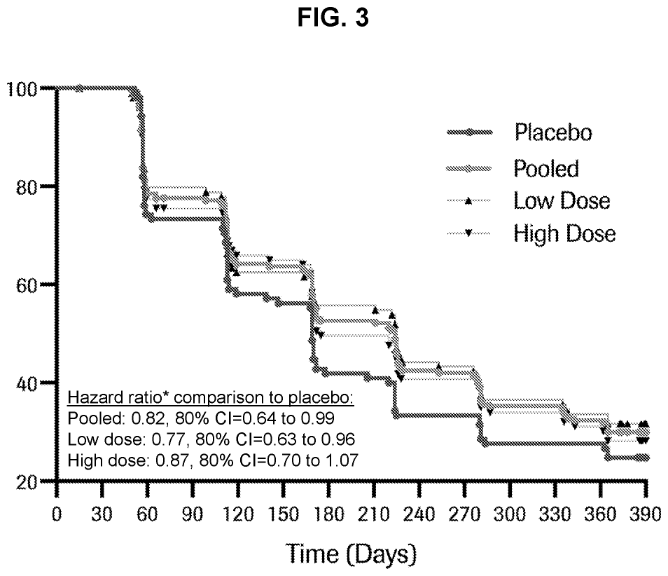

Furthermore, prasinezumab treatment resulted in reduced disease progression in prasinezumab-treated patients as demonstrated by a delay in time to clinically meaningful worsening of motor progression on the site rated assessment of time to at least a 5-point progression on MDS-UPDRS Part III versus placebo over one year, with a hazard ratio of 0.82 (FIG. 3).

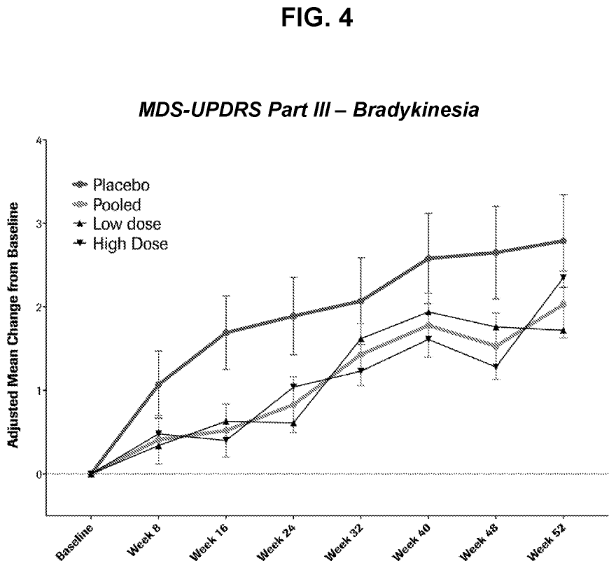

Signals of efficacy were observed on change from baseline on bradykinesia in prasinezumab-treated patients versus placebo at 52 weeks by site rating (pooled dose levels: −27.0%, −0.75, 80% CI=(−1.62, 0.11); low dose level: −38.3%, −1.07, 80% CI=(−2.07, −0.07); and high dose level: −15.7%, −0.44, 80% CI=(−1.45, 0.56)) (FIG. 4). Bradykinesia is one of the cardinal symptoms of Parkinson's disease and is assessed as a component of the MDS-UPDRS Part III clinical motor examination.

### Example 3. Passive Monitoring of Early-Stage Parkinson's Disease Patient Mobility in Phase I Alpha-Synuclein Antibody Clinical Trial with Smartphone Sensors

Gait and mobility in early-stage Parkinson's disease (PD) patients were measured using smartphone-based passive monitoring. In the Multiple Ascending Dose clinical trial of an alpha-synuclein antibody with a heavy chain variable region designated SEQ ID NO:10 and a light chain variable region designated SEQ ID NO:9, 44 PD patients and 35 age- and gender-matched healthy individuals performed smartphone-based assessments for up to 24 weeks and up to 6 weeks respectively. (Lipsmeier, F., et al., Mov Disord. 2017; 32 (suppl 2); W. Y. Cheng et al., 2017 IEEE/ACM International Conference on Connected Health: Applications, Systems and Engineering Technologies (CHASE), Philadelphia, P A, 2017, pp. 249-250).

For “passive monitoring”, subjects carried the smartphone with them as part of their daily routine, while sensors in the smartphone recording movement data continuously. In total, over 30,000 hours of passive monitoring data were collected. To classify the sensor signal into activity profiles, a Human Activity Recognition (HAR) model was built using Deep Neural Networks (DNN) trained on previously published data. The activity profiles of the participants determined by the HAR model showed significant differences between PD patients and healthy controls in the percentage of time walking and frequency in which subjects changed positions (sitting and standing).

The analysis solely focused on exploring differences between HC and PD cohorts, and did not look at antibody-related effects. In total, 24,104 hours of passive monitoring data were recorded for the PD cohort, and 8,614 hours for the HC cohort. In line with the approach of Rai, A. et al., (MobiCom′ 12, Aug. 22-26, 2012), accelerometer data was filtered out where the standard deviation of Euclidean norm less than 0.03 m/s2 more than 30 minutes, as during these spans smartphones were likely not carried by the subjects. This step removed 14% of the passive monitoring data.

A 9-layer neural network model structure was used. Similar structures have been used previously for HAR and have been shown to out-perform the traditional machine learning methods (F. J. Ordonez and D. Roggen, Sensors 2016, 16, 115). The HAR model was trained on two public data sets (G. M. Weiss and J. W. Lockhart, Proceedings of the AAAI-12 Workshop on Activity Context Representation: Techniques and Languages, Toronto, C A. 2012; A. Stisen, et al., 13th ACM Conference on Embedded Networked Sensor Systems, Seoul, Korea, 2015) to classify six activities: walking, stairs, jogging, sitting, standing, and lying down. The continuous accelerometer data were down-sampled into 20 Hz and segmented into 4-second windows with 75% overlapping with adjacent ones.

### A. Human Activity Recognition Performance Validation:

To ensure the HAR model can accurately translate the sensor data into activity profile, the performance of the model was first analyzed in the held-out validation set. The HAR model was able to correctly distinguish gait activities (walking, stairs, jogging) from stationary activities (sitting, standing, lying down) with more than 98% of accuracy. Additional validation on labeled Gait and Balance data from the trial data also showed that the HAR model was able to successfully profile the Gait segments with 96.9% of accuracy, and Balance segments with 99.5% accuracy.

### B. Activity Profiles Comparison

The mobility of each subject was quantified by calculating the proportion of time when the subject engaged in gait activities (walking, stairs, jogging) over the total passive monitoring coverage time of the patient. The overall proportion of different gait activities over the total coverage for PD and HC cohorts was calculated. In the PD cohort a median was detected of 9.7% of gait spans over all coverage spans as opposed to HC cohort's 15.1%. The HC cohort had a significantly higher per-subject gait activity level than PD cohort, with Mann-Whitney test P value 2.43E-8.

### Number of Sit-to-Stand and Stand-to-Sit Comparison

It has been observed that one manifestation of the functional impact of PD is in the sit-to-stand and stand-to-sit (STS) events (A. Zijlstra, et al., J. NeuroEngineering and Rehabilitation 2012, 9:75). From the activity profile, the coverage-normalized STS events were calculated for each subject. Median number of STS per hour of PD patient of 1.44 was observed, which was significantly lower than HC subject's 1.74. Mann-Whitney test P value between two groups was 1.60E-8.

Results from this study reflected that it is feasible to measure gait and mobility in early-stage PD patients using smartphone-based passive monitoring. Sensor data collected during passive monitoring provides previously inaccessible, ecologically valid insights into patients' daily behavior and functioning. Significant differences were observed between PD patients and healthy controls (HC).

### Example 4: 52 Week Study Monitoring of Emergence of Slopes of Motor Sign Progress in PD Patients

The study in Example 1 included monitoring of emergence of slopes of passive and active motor sign progress in PD patients.

Using a smart phone, a total of seventeen pre-specified sensor features were measured in the patient populations identified in Table 1 on a bi-weekly bases for 52 weeks. Sensor features aggregated (median) over all data points within each two-week window over the entire 52 weeks of study. If data for less than three features were collected per two week period, then the patient data was identified as missing.

Features were monitored one per task/side from active and passive monitoring. The sensor features include the following: (a) median gesture power of passively monitored gestures: (b) median turn speed in U-turn test and passively monitored gait, (c) jerk in balance test, (d) mel frequency cepstrum 2 in speech test, (e) voice jitter in sustained phonation, (f) number correct in Symbol Digit Modalities Test. For for least and most affected sides separately, the following features were monitored (g) speeded tapping variability, (h) maximum speed of hand-turning, (i) spiral celerity in draw-a-shape task, and (j) median squared energy in rest and postural tremor tasks.

Data was censored at the start of symptomatic PD treatment and the Prasinezumab treatment groups were combined (“pooled”). Linear mixed effect (LME) model with random slope (per two weeks) of change from baseline were determined. Covariates include baseline MAO-Bi therapy Yes/No; Age; Sex; and baseline DaT-SPECT Specific Binding Ratio in ipsilateral putamen. Alpha=0.2, beta=0.8; multiple comparison correct=15% False Discovery Rate (FDR). Non-normally distributed residuals, report Mixed Models with Repeated Measures (MMRM).

Table 3 reflects that Prasinezumab treatment is associated with decreased progression of upper limb bradykinesia.

TABLE 3

| Category | Source | Feature | Result |
|---|---|---|---|
| Bradykinesia | Speeded tapping test | Variability, L * | Effect favors |
| | | | prasinezumab |
| | | Variability, M | Effect favors |
| | | | prasinezumab |
| | Hand turning test | Maximum speed, L | No effect |
| | | Maximum speed, M | Effect favors |
| | | | prasinezumab |
| | Passive gestures | Power * | Effect favors |
| | | | prasinezumab |
| | Draw-a-shape | Spiral celerity, L | Effect favors |
| | | | placebo |
| | | Spiral celerity, M | No effect |
| Gait, balance | U-turn test | Median turn speed | Effect favors |
| | | | prasinezumab |
| | Passive turning | Median turn speed | No effect |
| | Balance test | Jerk | No effect |
| Tremor | Postural tremor test | Median squared energy, L | No effect |
| | | Median squared energy, M | No effect |
| | Rest tremor test | Median squared energy, L | No effect |
| | | Median squared energy, M | No effect |
| Speech | Free speech test | Mel frequency cepstrum 2 | No effect |
| | Sustained phonation test | Voice jitter | No effect |
| Cognition | SDMT | Number correct | No effect |

L = less affected side
M = most affected side
Celerity = accuracy/speed
* = false discovery rate (FDR) less than or equal to 2

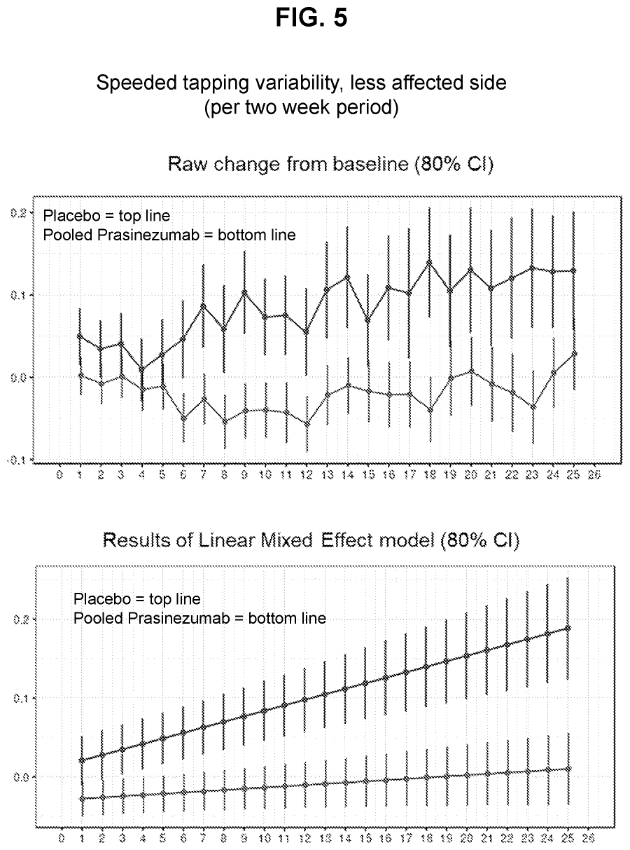

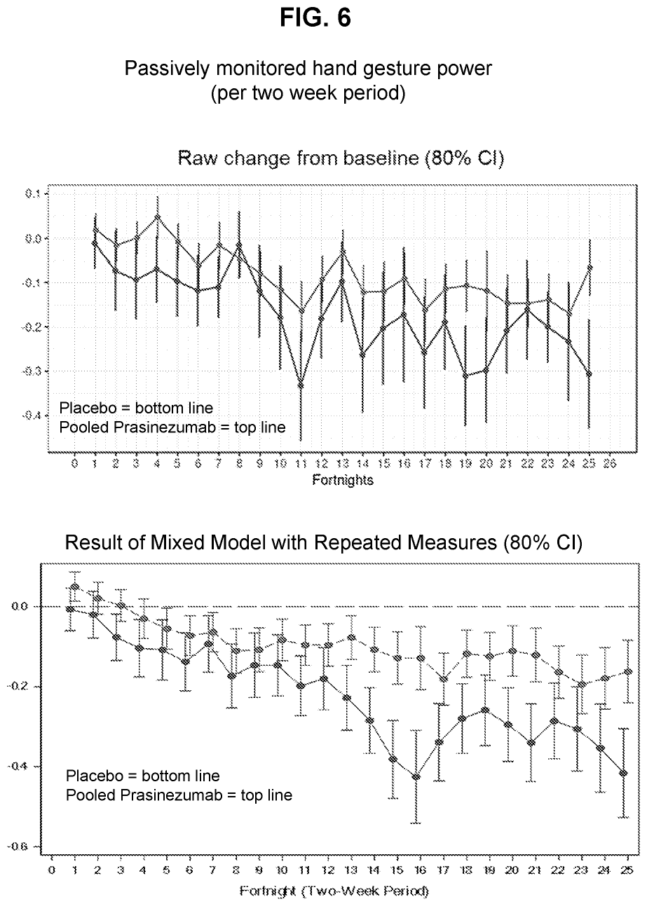

These results show that daily quantification via a mobile application in early-stage PD can demonstrate a divergence of slopes in bradykinesia progression. FIGS. 5 and 6 show that patients treated with Prasinezumab had less bradykinesia progress than those treated with placebo. FIG. 5 shows the monitoring results for speed tapping variability on the least affected side (FDR less than or equal to 2). FIG. 6 shows the results of passively monitoring hand gesture power (FDR less than or equal to 2). These results are consistent with other measures for evaluating motor function (e.g., MDS-UPDRS part III) that showed patients treated with prasinezumab had slowing of PD progression (e.g., maintained or had a slowing decline of motor function). As a result, these methods of monitoring motor function can be used to monitor patients treated with prasinezumab.

These results show that DHTT measurements of the core signs of PD remotely, continuously and objectively can enable the modelling of slopes of motor sign progression.

### Example 5: Generation and Analysis of PASADENA Digital Motor Score

From the study in Example 1, results from patients completing daily motor tests on a smartphone, utilizing input surfaces (e.g., screen) and internal sensors to assess measures of bradykinesia, (tremor/bradykinesia, tremor alone, rigidity and postural instability, and cognition) were combined to generate a “Digital PASADENA Motor Score”. The frequent testing enabled modeling of the slopes of motor progression reflecting primarily bradykinesia measures. This mixed model demonstrated a reduced motor progression as measured by the digital PASADENA Motor Score in both the low and high dose groups (compared to placebo).

In the pooled total population, a reduced decline in PASADENA Digital Motor Score of 25.0% versus placebo over one year of treatment was observed. Low dose effects appeared more robust at a 30.3% reduction in decline; whereas, the higher dose demonstrated a 21.5% reduction in decline at one year (see FIG. 8A; pooled dose levels: −25.0%, −0.030, 80% CI=(−0.050, −0.010); low dose level: −30.3%, −0.040, 80% CI=(−0.063, −0.017); high dose level: −21.5%, −0.029, 80% CI=(−0.052, −0.006)).

In the MAO-B inhibitor treated subgroup, a reduced decline in PASADENA Digital Motor Score of 26.0% versus placebo over one year of treatment was observed. Low dose effects appeared more robust at a 31.0% reduction in decline; whereas, the higher dose demonstrated a 20.9% reduction in decline at one year (see FIG. 8B; pooled dose levels: −26.0%, −0.032, 80% CI=(−0.062, −0.003); low dose level: −31.0%, −0.039, 80% CI=(−0.072, −0.049); high dose level: −20.9%, −0.026, 80% CI=(−0.060, 0.008)).

In the diffuse malignant subgroup, a reduced decline in PASADENA Digital Motor Score of 35.7% versus placebo over one year of treatment was observed. Low dose effects appeared less robust at a 25.2% reduction in decline; whereas, the high dose demonstrated a 46.2% reduction in decline at one year (see FIG. 8C; pooled dose levels: −35.7%, −0.055, 80% CI=(−0.105, −0.005); low dose level: −25.2%, −0.039, 80% CI=(−0.094, 0.017); high dose level: −46.2%, −0.071, 80% CI=(−0.126, −0.017)).

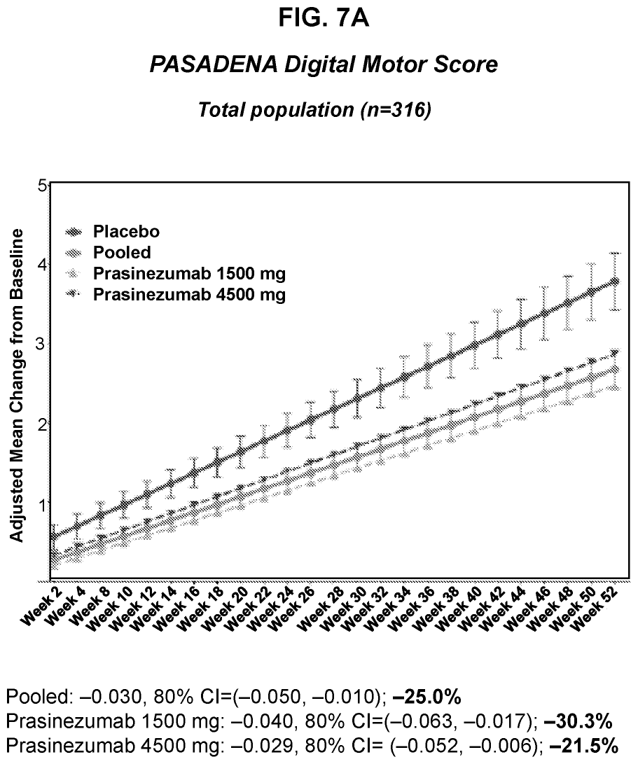

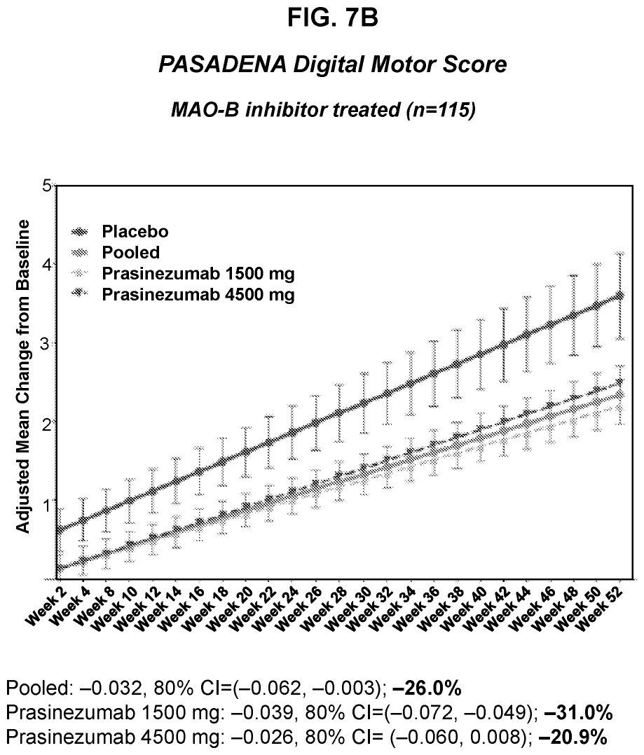

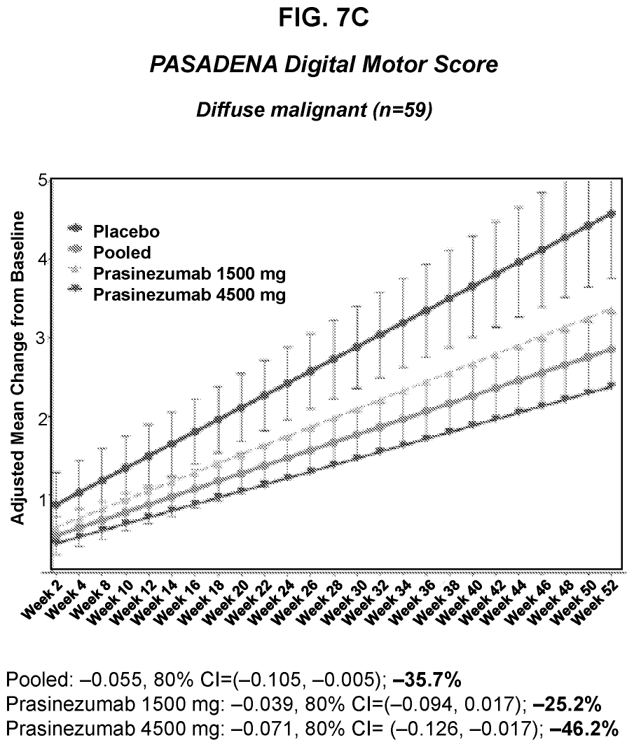

As shown in FIGS. 7A-7C, patients who started symptomatic PD treatment contribute until the last visit before symptomatic PD treatment is started. Bars represent 80% CI. Estimates are based on a MMRM with the following covariates: MAO-B inhibitor at baseline (yes/no), treatment, week, age<60 vs ≥60, sex, DaT-SPECT putamen binding ratio (contralateral to most clinically affected side), baseline MDS-UPDRS corresponding endpoint. Pooled-dose analysis is a pre-specified exploratory analysis. 4500 mg for ≥65 kg; 3500 mg for <65 kg.

SEQUENCES

SEQ ID NO: 1 is an Hu9E4VLv3 variable region.
DIQMTQSPSSLSASVGDRVTITCKSIQTLLYSSNQKNYLAWFQQKPGKAPKLLIYWA
SIRKSGVPSRFSGSGSGTDFTLTISSLQPEDLATYYCQQYYSYPLTFGGGTKLEIK
SEQ ID NO: 2 is an Hu9E4VLvl variable region.
DIQMTQSPSSLSASVGDRVTITCKSIQTLLYSSNQKNYLAWFQQKPGKAPKLLIYWA
SIRKSGVPSRFSGSGSGTDFTLTISSLQPEDFATYYCQQYYSYPLTFGGGTKLEIK
SEQ ID NO: 3 is an Hu9E4VLv2 (No backmutation) variable region.
DIQMTQSPSSLSASVGDRVTITCKSIQTLLYSSNQKNYLAWYQQKPGKAPKLLIYWA
SIRKSGVPSRFSGSGSGTDFTLTISSLQPEDFATYYCQQYYSYPLTFGGGTKLEIK
SEQ ID NO: 4 is an Hu9E4VHv3 variable region.
EVQLVESGGGLVQPGGSLRLSCAASGFTFSNYGMSWVRQAPGKGLEWVASISSGGGS
TYYPDNVKGRFTISRDDAKNSLYLQMNSLRAEDTAVYYCARGGAGIDYWGQGTLVTV
SS
SEQ ID NO: 5 is an Hu9E4VHv1 variable region.
EVQLVESGGGLVQPGGSLRLSCAASGFTFSNYGMSWVRQAPGKGLEWVASISSGGGS
TYYPDNVKGRFTISRDDAKNSLYLQMNSLRAEDTAVYYCSRGGAGIDYWGQGTLVTV
SS
SEQ ID NO: 6 is an Hu9E4VHv2 variable region.
EVQLVESGGGLVQPGGSLRLSCAASGFTFSNYGMSWVRQAPGKGLEWVASISSGGGS
TYYPDNVKGRFTISRDNAKNSLYLQMNSLRAEDTAVYYCSRGGAGIDYWGQGTLVTV
SS
SEQ ID NO: 7 is an Hu9E4VHv4 (no backmutation) variable region.
EVQLVESGGGLVQPGGSLRLSCAASGFTFSNYGMSWVRQAPGKGLEWVASISSGGGS
TYYPDNVKGRFTISRDNAKNSLYLQMNSLRAEDTAVYYCARGGAGIDYWGQGTLVTV
SS
SEQ ID NO: 8 is the amino acid sequence of natural human wildtype alpha-
synuclein.
MDVFMKGLSKAKEGVVAAAEKTKQGVAEAAGKTKEGVLYVGSKTKEGVVHGVATVAE
KTKEQVTNVGGAVVTGVTAVAQKTVEGAGSIAAATGFVKKDQLGKNEEGAPQEGILE
DMPVDPDNEAYEMPSEEGYQDYEPEA
SEQ ID NO: 9 is the amino acid sequence of Prasinezumab light chain

| | |
|---|---|
| DIQMTQSPSSLSASVGDRVTITCKSIQTLLYSSNQKNYLAWFQQKPGKAPKLLIYWASIR | 60 |
| KSGVPSRFSGSGSGTDFTLTISSLQPEDLATYYCQQYYSYPLTFGGGTKLEIKRTVAAPS | 120 |
| VFIFPPSDEQLKSGTASVVCLLNNFYPREAKVQWKVDNALQSGNSQESVTEQDSKDSTYS | 180 |
| LSSTLTLSKADYEKHKVYACEVTHQGLSSPVTKSENRGEC | 220 |

SEQ ID NO: 10 is the amino acid sequence of Prasinezumab heavy chain

| | |
|---|---|
| EVQLVESGGGLVQPGGSLRLSCAASGFTFSNYGMSWVRQAPGKGLEWVASISSGGGSTYY | 60 |
| PDNVKGRFTISRDDAKNSLYLQMNSLRAEDTAVYYCARGGAGIDYWGQGTLVTVSSASTK | 120 |
| GPSVFPLAPSSKSTSGGTAALGCLVKDYFPEPVTVSWNSGALTSGVHTFPAVLQSSGLYS | 180 |
| LSSVVTVPSSSLGTQTYICNVNHKPSNTKVDKRVEPKSCDKTHTCPPCPAPELLGGPSVF | 240 |
| LFPPKPKDTLMISRTPEVTCVVVDVSHEDPEVKFNWYVDGVEVHNAKTKPREEQYNSTYR | 300 |
| VVSVLTVLHQDWLNGKEYKCKVSNKALPAPIEKTISKAKGQPREPQVYTLPPSREEMTKN | 360 |
| QVSLTCLVKGFYPSDIAVEWESNGQPENNYKTTPPVLDSDGSFFLYSKLTVDKSRWQQGN | 420 |
| VFSCSVMHEALHNHYTQKSLSLSPGK | 446 |

What is claimed is:

1. A method for monitoring the motor function of a patient that has Parkinson's Disease (PD) or is at risk for PD who has been administered Prasinezumab, the method comprising.
(a) providing the patient with a mobile device programmed to receive and transmit data acquired from sensors internal and/or external to the mobile device that measure passive and/or active movement of the patient or a mobile device application programmed to receive and transmit data acquired from sensors internal and/or external to the mobile device that measure passive and/or active movement of the patient;
(b) collecting data transmitted from the mobile device; and
(c) comparing the data acquired from the patient with control data to assess presence or extent of movement deficits in the subject and/or monitoring the data acquired from the patient for a period of time sufficient to identify changes in the patient's active or passive motor function.

2. The method of claim 1, wherein the sensors transmit data acquired from active movement of the patient.

3. The method of any of claim 1 or 2, wherein the mobile device is programmed to receive and transmit data from external sensors attached to upper and lower limbs of the patient.

4. The method of any of claims 1-3, wherein the mobile device acquires data from sensors on the upper and lower limbs of the subject.

5. The method of any of claims 1-4, wherein the mobile device is carried by the subject and acquires data from an internal sensor.

6. The method of any of claims 1-5, wherein the movement comprises tapping the device, sitting and standing.

7. The method of any of claims 1-5, wherein the sensors measure one or more of the following features of the patient's movement:
(a) median gesture power of passively monitored gestures:
(b) median turn speed in U-turn test and passively monitored gait,
(c) jerk in balance test,
(d) mel frequency cepstrum 2 in speech test,
(e) voice jitter in sustained phonation,
(f) number correct in Symbol Digit Modalities Test.
(g) speeded tapping variability,
(h) maximum speed of hand-turning,
(i) spiral celerity in draw-a-shape task, and
(j) median squared energy in rest and postural tremor tasks.

8. The method claim 7, wherein the independently measure movement from the least affected side and the most affected side of the patient.

9. The method of any of claims 1-8, wherein the data collected from the device is compared the patient's MDS-UPDRS score.

10. The method of claim 9, wherein the MDS-UPDRS score comprises of one of MDS-UPDRS Part I, MDS-UPDRS Part II, or UPDRS Part III.

11. The method of claim 10, wherein the MDS-UPDRS score comprises UPDRS Part III.

12. The method of any one of claims 1-11, further comprising administering a regimen Prasinezumab to the patient.

13. The method of any one of claims 1-12, wherein the regimen of Prasinezumab comprises 1000-5000 mg of Prasinezumab at intervals of 3 to 5 weeks.

14. The method of claim 13, wherein Prasinezumab is administered intravenously.

15. The method of any of claims 1-14, further comprising administering to the patient a MAO-B inhibitor.

16. The method of any of claims 1-15, wherein the patient is treatment naïve, was diagnosed as having PD in the last two years, or was previously treated with a MAO-B inhibitor.

17. The method of any of claims 1-16, wherein the patient has a weight greater than 65 kg and is administered a dose of 4500 mg Prasinezumab once every 4 weeks.

18. The method of any of claim 1-16, wherein the patient has a weight less than 65 kg and is administered a dose of 3500 mg Prasinezumab once every 4 weeks.

19. The method of any of claims 1-16, wherein the patient is administered a dose of 1500 mg antibody every 4 weeks.

20. The method of any of claims 1-19, wherein the patient is administered Prasinezumab once every 4 weeks for at least 52 weeks.

21. The method of any of claim 1-20 wherein the period of time sufficient to identify changes in the patient's active or passive motor function comprises 4-52 weeks.

22. The method of claim 21, wherein the period of time is 4 weeks, 8 weeks, 16 weeks, 20 weeks, 24 weeks, 28 weeks, 32 weeks, 36 weeks, 42 weeks, 46 weeks or 52 weeks.


SEQUENCE LISTING (10 sequences)

SEQ ID NO: 1 - protein, 113 aa, Artificial Sequence
  Hu9E4VLv3 variable region
DIQMTQSPSSLSASVGDRVTITCKSIQTLLYSSNQKNYLAWFQQKPGKAPKLLIYWASIRKSGVPSRFSGSGSGTDFTLTISSLQPEDLATYYCQQYYSYPLTFGGGTKLEIK

SEQ ID NO: 2 - protein, 113 aa, Artificial Sequence
  Hu9E4VLv1 variable region
DIQMTQSPSSLSASVGDRVTITCKSIQTLLYSSNQKNYLAWFQQKPGKAPKLLIYWASIRKSGVPSRFSGSGSGTDFTLTISSLQPEDFATYYCQQYYSYPLTFGGGTKLEIK

SEQ ID NO: 3 - protein, 113 aa, Artificial Sequence
  Hu9E4VLv2 (No backmutation) variable region
DIQMTQSPSSLSASVGDRVTITCKSIQTLLYSSNQKNYLAWYQQKPGKAPKLLIYWASIRKSGVPSRFSGSGSGTDFTLTISSLQPEDFATYYCQQYYSYPLTFGGGTKLEIK

SEQ ID NO: 4 - protein, 116 aa, Artificial Sequence
  Hu9E4VHv3 variable region
EVQLVESGGGLVQPGGSLRLSCAASGFTFSNYGMSWVRQAPGKGLEWVASISSGGGSTYYPDNVKGRFTISRDDAKNSLYLQMNSLRAEDTAVYYCARGGAGIDYWGQGTLVTVSS

SEQ ID NO: 5 - protein, 116 aa, Artificial Sequence
  Hu9E4VHv1 variable region
EVQLVESGGGLVQPGGSLRLSCAASGFTFSNYGMSWVRQAPGKGLEWVASISSGGGSTYYPDNVKGRFTISRDDAKNSLYLQMNSLRAEDTAVYYCSRGGAGIDYWGQGTLVTVSS

SEQ ID NO: 6 - protein, 116 aa, Artificial Sequence
  Hu9E4VHv2 variable region
EVQLVESGGGLVQPGGSLRLSCAASGFTFSNYGMSWVRQAPGKGLEWVASISSGGGSTYYPDNVKGRFTISRDNAKNSLYLQMNSLRAEDTAVYYCSRGGAGIDYWGQGTLVTVSS

SEQ ID NO: 7 - protein, 116 aa, Artificial Sequence
  Hu9E4VHv4 (no backmutation) variable region
EVQLVESGGGLVQPGGSLRLSCAASGFTFSNYGMSWVRQAPGKGLEWVASISSGGGSTYYPDNVKGRFTISRDNAKNSLYLQMNSLRAEDTAVYYCARGGAGIDYWGQGTLVTVSS

SEQ ID NO: 8 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 9 - protein, 220 aa, Artificial Sequence
  Prasinezumab light chain
DIQMTQSPSSLSASVGDRVTITCKSIQTLLYSSNQKNYLAWFQQKPGKAPKLLIYWASIRKSGVPSRFSGSGSGTDFTLTISSLQPEDLATYYCQQYYSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 446 aa, Artificial Sequence
  Prasinezumab heavy chain
EVQLVESGGGLVQPGGSLRLSCAASGFTFSNYGMSWVRQAPGKGLEWVASISSGGGSTYYPDNVKGRFTISRDDAKNSLYLQMNSLRAEDTAVYYCARGGAGIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK